(19) United States
(12) Patent Application Publication
(10) Pub. No.: US 2020/0305847 A1
(43) Pub. Date: Oct. 1, 2020

(54) METHOD AND SYSTEM THEREOF FOR RECONSTRUCTING TRACHEA MODEL USING COMPUTER-VISION AND DEEP-LEARNING TECHNIQUES

(71) Applicant: Fei-Kai Syu, Pingtung City (TW)

(72) Inventor: Fei-Kai Syu, Pingtung City (TW)

(21) Appl. No.: 16/367,284

(22) Filed: Mar. 28, 2019

(51) Int. Cl. A61B 8/08; A61B 8/12; G06T 7/00; G06T 17/00

(52) U.S. Cl. CPC A61B 8/5207; A61B 8/12; G06T 7/0012; G06T 2210/41; G06T 2207/10012; G06T 2207/10136; G06T 2207/30061; G06T 17/00

(57) ABSTRACT

A tracheal model reconstruction method using the computer-vision and deep-learning techniques; which comprises the following steps: obtaining an image of the tracheal wall, loading the graph-information, processing the image, extracting the image-feature, comparing the image, estimating the position-pose and converting the spatial-information, and reconstructing a three-dimensional trachea model. Thereby, providing a tracheal model reconstruction method that can correctly and quickly reconstruct and record a stereoscopic three-dimensional tracheal model.

### (a) TECHNICAL FIELD OF THE INVENTION

The present invention is a tracheal model reconstruction method and the system thereof using the computer-vision and deep-learning techniques, and especially relates to a tracheal model reconstruction method and the system thereof capable of correctly and quickly reconstructing and recording a stereoscopic three-dimensional trachea model.

### (b) DESCRIPTION OF THE PRIOR ART

When the patient underwent the general anesthesia, the cardiopulmonary resuscitation, or the patient is unable to breathe on their own during surgery, the patient must be intubated to insert the artificial airway into the trachea; so that the medical gas is smoothly delivered into the patient's trachea.

When intubating treatment, because the medical staff can not directly visualize and adjust the artificial airway, it can only rely on the medical staffs touch and past experience to avoid stabbing the patient's trachea; so it needs to take several operations to be successful, and it will delay the time for establishing a smooth airway.

Therefore, the rapid and correct establishment of a three-dimensional trachea model for providing the medical personnel to assist intubation is an urgent problem to be solved.

### SUMMARY OF THE INVENTION

The object of the present invention is to improve the above-mentioned defects, and to provide a tracheal model reconstruction method and system thereof capable of correctly and quickly reconstructing and recording a stereoscopic three-dimensional trachea model.

In order to achieve the above object, the trachea model reconstruction method using computer-vision and deep-learning techniques of the present invention comprises the following steps:

obtaining an image of the tracheal wall: the endoscope lens is used to shoot and extract a continuous image of the oral cavity to the trachea;

loading the graph-information: loading and storing the continuous image shot and extracted by the endoscope lens for subsequent processing;

processing the image: de-noise and noise reduction are performed on the continuous image shot and extracted and the image enhancement is processed to emphasize the image details for obtaining a clear image;

extracting the image-feature: the feature extraction method of regional extremum is applied to the continuous image after being processed by the step of processing the image for extracting and filtering the feature-points; and then the feature-points after being extracted and filtered are stored;

comparing the image: compare the image feature-points of two successive connected images after being processed by the step of extracting the image-feature to find out the common feature-points and record and store;

estimating the position-pose and converting the spatial-information: the common image feature-points are used to achieve assisting recognition by using the deep-learning, and then estimating the position and pose of the endoscope lens reaching in the trachea in the three-dimensional space when the endoscope lens shoots the common image feature-points; and then they are converted and calculated to the spatial-information of the depth and angle of the endoscope lens when extending into the trachea to shoot; and

reconstructing a three-dimensional trachea model: the common image feature-points after being processed by the step of comparing the image are projected into the three-dimensional space; which the spatial-information of the shooting depth and angle of the endoscope lens obtained in the step of estimating the position-pose and converting the spatial-information is collaborated with the common image feature-points to reconstruct and record as an actual stereoscopic three-dimensional trachea model.

By the above method, the three-dimensional trachea model can be quickly and correctly reconstructed and formed, and further assisting the personnel to intubate.

Thereby, the present invention provides a tracheal model reconstruction method that can correctly and quickly reconstruct and record a stereoscopic three-dimensional tracheal model for providing the subsequent medical research or use.

### BRIEF DESCRIPTION OF THE DRAWINGS

FIG. 1 is a step flow chart of the present invention.

FIG. 2 is a system block diagram of the present invention.

FIG. 3 is a system block diagram of the present invention combined with an endoscope lens.

### DETAILED DESCRIPTION OF THE PREFERRED EMBODIMENTS

The following descriptions are exemplary embodiments only, and are not intended to limit the scope, applicability or configuration of the invention in any way. Rather, the following detailed description provides a convenient illustration for implementing exemplary embodiments of the invention. Various changes to the described embodiments may be made in the function and arrangement of the elements described without departing from the scope of the invention as set forth in the appended claims.

The foregoing and other aspects, features, and utilities of the present invention will be best understood from the following detailed description of the preferred embodiments when read in conjunction with the accompanying drawings.

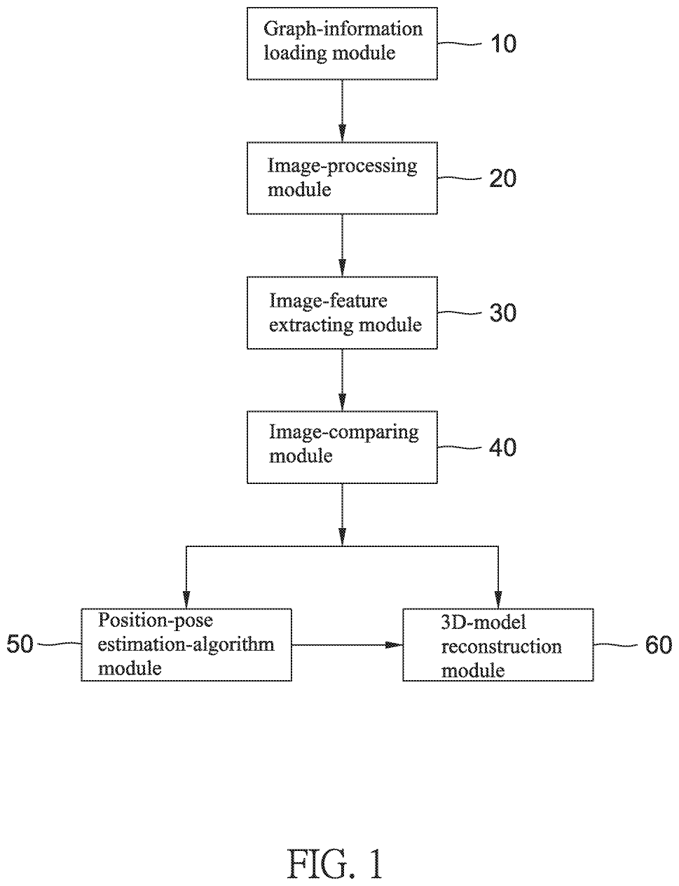

Regarding the technical means and the structure applied by the present invention to achieve the object, the embodiment shown in FIG. 1 to FIG. 3 will be explained in detail as follows; as shown in FIG. 1, the trachea model reconstruction method using computer-vision and deep-learning techniques in the embodiment comprises the following steps.

Obtaining an image of the tracheal wall: The endoscope lens 70 is used to shoot and extract a continuous image of the oral cavity to the trachea.

Loading the graph-information: Loading and storing the continuous image shot and extracted by the endoscope lens 70 for subsequent processing.

Processing the image: De-noise and noise reduction are performed on the continuous image shot and extracted and the image enhancement is processed to emphasize the image details for obtaining a clear image.

Extracting the image-feature: The feature extraction method (such as SIFT, SURF, ORB, . . . , etc.) of regional extremum is applied to the continuous image after being processed by the step of processing the image for extracting and filtering the feature-points; and then the feature-points after being extracted and filtered are stored.

Comparing the image: Compare the image feature-points of two successive connected images after being processed by the step of extracting the image-feature to find out the common feature-points and record and store.

Estimating the position-pose and converting the spatial-information: The common image feature-points are used to achieve assisting recognition by using the deep-learning, and then estimating the position and pose of the endoscope lens 70 reaching in the trachea in the three-dimensional space when the endoscope lens 70 shoots the common image feature-points; and then they are converted and calculated to the spatial-information of the depth and angle of the endoscope lens 70 when extending into the trachea to shoot.

Reconstructing a three-dimensional trachea model: The common image feature-points after being processed by the step of comparing the image are projected into the three-dimensional space; which the spatial-information of the shooting depth and angle of the endoscope lens 70 obtained in the step of estimating the position-pose and converting the spatial-information is collaborated with the common image feature-points to reconstruct and record as an actual stereoscopic three-dimensional trachea model.

By the above method, the three-dimensional trachea model can be quickly and correctly reconstructed and formed, and further assisting the personnel to intubate.

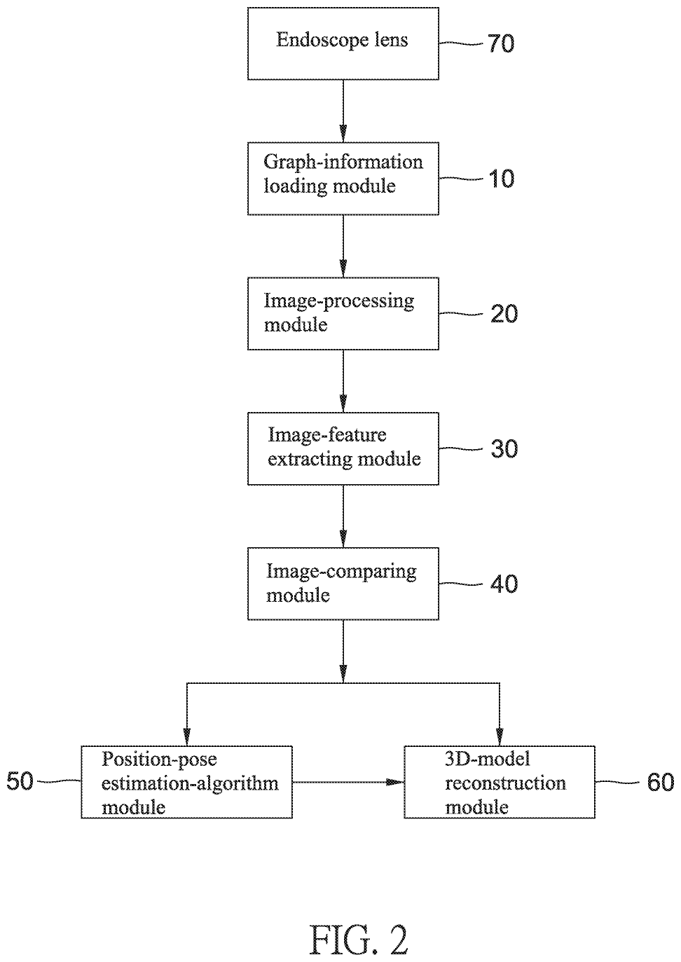

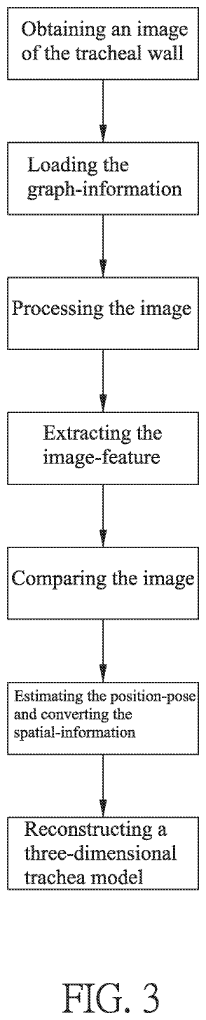

n order to achieve the above method, the model reconstruction system of the present invention is further explained in detail with the embodiment shown in FIG. 2 to FIG. 3 as follows.

As shown in FIG. 2, the trachea model reconstruction system using computer-vision and deep-learning techniques of the present invention comprises a graph-information loading module 10, an image-processing module 20, an image-feature extracting module 30, an image-comparing module 40, a position-pose estimation-algorithm module 50, and a 3D-model reconstruction module 60; which are further described in detail as follows.

The graph-information loading module 10 (please simultaneously refer to FIG. 3) is connected with the endoscope lens 70 and for loading and storing the continuous image which is shot and extracted by the endoscope lens 70 entering the trachea from the oral cavity to provide for the subsequent processing.

The image-processing module 20 (please simultaneously refer to FIG. 3) is connected with the graph-information loading module 10 for receiving the continuous image loaded by the graph-information loading module 10; and is for processing the denoise and noise-decreasing of the continuous image; and using the image enhancement technique to emphasize the image details to obtain a clear image.

The image-feature extracting module 30 (please simultaneously refer to FIG. 3) is connected with the image-processing module 20, and is for extracting and filtering the feature-points of the clear image after being processed by the image-processing module 20 through the feature extraction method of the regional extremum; and then stores the feature-points after being extracted and filtered.

Continuing to the above description, the feature extraction method of the regional extremum may be Scale-Invariant Feature Transform (SIFT), Speeded Up Robust Features (SURF), fast feature-point extraction and description (Oriented FAST and Rotated BRIEF, referred to as ORB), and other methods.

The image-comparing module 40 (please simultaneously refer to FIG. 3) is connected with the image-feature extracting module 30, and is for receiving the image feature-points extracted and filtered by the image-feature extracting module 30; and then comparing the image feature-points of two successive connected images to find out the common feature-points, and then recording and storing.

The position-pose estimation-algorithm module 50 (please simultaneously refer to FIG. 3) having the function of deep-learning is connected with the image-comparing module 40, and is for receiving the common feature-points found by the image-comparing module 40; at the same time, using the deep-learning model to achieve assisting identification; and then estimating the position and pose of the endoscope lens 70 reaching in the trachea in the three-dimensional space when the endoscope lens 70 shoots and extracts image; and then they are converted and calculated to the spatial-information of the depth and angle of the endoscope lens 70 when extending into the trachea to shoot image.

The 3D-model reconstruction module 60 (please simultaneously refer to FIG. 3) is connected with the image-comparing module 40 and the position-pose estimation-algorithm module 50 for receiving the common image feature-points found by the image-comparing module 40, and is for receiving the spatial-information converted and calculated by the position-pose estimation-algorithm module 50; thereby projecting the common image feature-points into the three-dimensional space; which the common image feature-points and the spatial-information are collaborated to reconstruct and record as an actual stereoscopic three-dimensional trachea model.

In addition, in the estimating the position-pose and converting the spatial-information step and the position-pose estimation-algorithm module 50, a plurality of patients' tracheal image data are shot and extracted to capture the image feature-points; and input the image feature-points and the shooting images into the deep-learning model; which the deep-learning model can be selected from the group consisting of supervised learning, unsupervised learning, semi-supervised learning, and reinforced learning (e.g., neural networks, random forest, support vector machine SVM, decision tree, or cluster, etc.); so that it can recognize the depth, angle, path position, path direction, and path trajectory for the endoscope lens 70 extending into the trachea; and it can recognize the characteristics and shape of the tracheal wall.

Therefore, the present invention uses the endoscope lens 70 to shoot a continuous image, and then denoises, reduces the noise, and enhances the image details; and then extracts the feature-points and compares the common feature-points; and then the position-pose estimation having the deep-learning function is used to capture the position and pose information of the continuous image; and further captures the depth and angle information of the endoscope lens 70 extending into the trachea; the movement trajectory of the endoscope lens 70 can be delineated; and the feature extraction method of the computer-vision and the visual distance measurement (Visual Odometry) can be realized and used to correctly and quickly reconstruct the stereoscopic three-dimensional tracheal model for providing the intubation assistance and the subsequent medical research or use.

I claim:

1. A trachea model reconstruction method using computer-vision and deep-learning techniques, which comprises the following steps:
obtaining an image of the tracheal wall: the endoscope lens is used to shoot and extract a continuous image of the oral cavity to the trachea;
loading the graph-information: loading and storing the continuous image shot and extracted by the endoscope lens for subsequent processing;
processing the image: de-noise and noise reduction are performed on the continuous image shot and extracted and the image enhancement is processed to emphasize the image details for obtaining a clear image;
extracting the image-feature: the feature extraction method of regional extremum is applied to the continuous image after being processed by the step of processing the image for extracting and filtering the feature-points; and then the feature-points after being extracted and filtered are stored;
comparing the image: compare the image feature-points of two successive connected images after being processed by the step of extracting the image-feature to find out the common feature-points and record and store;
estimating the position-pose and converting the spatial-information: the common image feature-points are used to achieve assisting recognition by using the deep-learning, and then estimating the position and pose of the endoscope lens reaching in the trachea in the three-dimensional space when the endoscope lens shoots the common image feature-points; and then they are converted and calculated to the spatial-information of the depth and angle of the endoscope lens when extending into the trachea to shoot; and
reconstructing a three-dimensional trachea model: the common image feature-points after being processed by the step of comparing the image are projected into the three-dimensional space; which the spatial-information of the shooting depth and angle of the endoscope lens obtained in the step of estimating the position-pose and converting the spatial-information is collaborated with the common image feature-points to reconstruct and record as an actual stereoscopic three-dimensional trachea model.

2. A trachea model reconstruction system using computer-vision and deep-learning techniques, which is applied to the trachea model reconstruction method using computer-vision and deep-learning techniques of claim 1 and comprises a graph-information loading module, an image-processing module, an image-feature extracting module, an image-comparing module, a position-pose estimation-algorithm module, and a 3D-model reconstruction module; wherein:
the graph-information loading module is connected with the endoscope lens and for loading and storing the continuous image which is shot and extracted by the endoscope lens entering the trachea from the oral cavity to provide for the subsequent processing;
the image-processing module is connected with the graph-information loading module for receiving the continuous image loaded by the graph-information loading module; and is for processing the denoise and noise-decreasing of the continuous image; and using the image enhancement technique to emphasize the image details;
the image-feature extracting module is connected with the image-processing module, and is for extracting and filtering the feature-points of the continuous image after being processed by the image-processing module through the feature extraction method of the regional extremum; and then stores the feature-points after being extracted and filtered;
the image-comparing module is connected with the image-feature extracting module, and is for receiving the image feature-points extracted and filtered by the image-feature extracting module; and then comparing the image feature-points of two successive connected images to find out the common feature-points, and then recording and storing;
the position-pose estimation-algorithm module having the function of deep-learning is connected with the image-comparing module and is for receiving the common feature-points found by the image-comparing module; at the same time, using the deep-learning model to achieve assisting identification; and then estimating the position and pose of the endoscope lens reaching in the trachea in the three-dimensional space when the endoscope lens shoots and extracts image; and then they are converted and calculated to the spatial-information of the depth and angle of the endoscope lens when extending into the trachea to shoot image; and
the 3D-model reconstruction module is connected with the image-comparing module and the position-pose estimation-algorithm module for receiving the common image feature-points found by the image-comparing module, and is for receiving the spatial-information converted and calculated by the position-pose estimation-algorithm module; thereby projecting the common image feature-points into the three-dimensional space; which the common image feature-points and the spatial-information are collaborated to reconstruct and record as an actual stereoscopic three-dimensional trachea model.
